(19) United States
(12) Patent Application Publication
(10) Pub. No.: US 2006/0239937 A2
(43) Pub. Date: Oct. 26, 2006

(54) STABLE FOAM CREAM

(75) Inventor: Fritz NEUBOURG, Emsdetten (DE)

Correspondence Address: STERNE, KESSLER, GOLDSTEIN & FOX PLLC, 1100 NEW YORK AVENUE, N.W., WASHINGTON, DC, 20005, US

(73) Assignee: Neubourg Skin Care GmbH and Co KG, Emsdetten (DE)

(21) Appl. No.: 10/538,666

(22) Filed: Sep. 12, 2005

Related U.S. Application Data
(60) Provisional application No. 60/432,632, filed on Dec. 12, 2002.

(30) Foreign Application Priority Data
Dec. 12, 2002 (DE) EP02027828.9

(51) Int. Cl. A61K 9/00

(52) U.S. Cl. 424/47

(57) ABSTRACT

Abstract of the Disclosure

The invention relates to a stable foam cream, containing lipophilic and hydrophilic components and a propellant gas, obtainable by subjecting the foam cream to heat treatment. The invention also relates to the production thereof.

### Detailed Description of the Invention

The invention relates to a stable foam cream, containing lipophilic and hydrophilic components and a propellant gas, obtainable by subjecting the foam cream to a heat treatment, as well as to methods for the manufacture of a stable foam cream.

The principle for preparing an emulsoid two-phase system comprising lipophilic or hydrophobic parts, respectively, on the one hand, and hydrophilic components on the other hand, for the manufacture of foam preparations is well known. Such foam preparations are utilized, inter alia, as skin creams. The foam is formed by foaming a basic preparation with a propellant gas. When using such foam preparations, a two-dimensional two-phase network is formed on the treated skin, the hydrophilic components bind to the keratin of the horn layer and allow for an evaporation of sweat, while the lipophilic parts inhibit the penetration of moisture at the skin surface. Such foam creams are, inter alia, described in documents WO-A99/08649 and WO-A-98/31339.

However, such foams in the form of aerosols are complex physico-chemical forms which cannot be manufactured at will. The stability of such foams is strongly dependent on the specific composition of the foam forming components. Even small deviations in the composition may lead to a collapse of the foam. "Collapsing" means that the foam, directly after its release from the tin, shows a strong formation of blisters and then collapses. This interferes with the positive properties of the foam (particularly its rapid absorption after application to the skin) and is consequently not accepted by the consumers. Therefore, a mixture of active agents per se cannot be formulated as a foam without any further ado. It is often observed that a normally stable foam tends to collapse upon addition of a further component. The manufacturer of such foams are generally well aware of the problem that upon manufacturing of well-tested stable foams, one batch is unexpectedly produced in which the foam collapses. Then, this batch has to be newly produced. If the problem is not already recognized by the manufacturer, the result is an unsatisfactory use by the customer. Collapsed or broken down foams cannot normally be recycled by the manufacturer and have to be disposed.

According to the prior art, this problem is solved in that specific manufacturing methods are utilized. WO 99/08649 discloses a technique, in which two separated phases I and II are provided in an exact protocol, pre-treated and mixed. The method comprises several steps which have to be carefully controlled by the manufacturer.

GB-A-2 204 875 relates to a method for realizing monocarboxylic acids. Finely divided particles are contacted with C₁₀-C₂₂ carboxylic acids in an aqueous medium with a neutralizing agent, for example ethanolamine, at a temperature below the melting point of said fatty acids. This results in pumpable liquid soap solutions containing 0.3 percent by weight to 40 percent by weight of soap. These are used for the manufacture of shaving foams by admixing C₃-or C₄-hydrocarbon propellant gases.

FR-A-2,217,405 relates to the preparation of foam creams in the form of oil-in-water emulsions.

WO-A-98/31339 relates to skin care products made by a two-phase system which is capable of producing a semi-permeable membrane on the skin.

It is an object of the invention to provide foams, which substantially do not collapse and which do not show the above-mentioned problems. Furthermore, methods should be provided to produce such foams and for making collapsed foams reusable.

Surprisingly, the problem underlying the invention is solved by a stable foam cream containing lipophilic and hydrophilic components and a propellant gas, obtainable by subjecting the foam cream to a heat treatment, characterized in that the treatment is conducted over a time period of at least 5, preferably 10 minutes, and/or 4 minutes to two hours at 40 to 75°C. In particular, the heat treatment is done after the addition of the propellant gas. Alternatively, the heat treatment is done by heating the propellant gas before and/or during its addition to the cream preparation. Both methods may be combined according to the invention.

The heat treatment after the addition of the propellant gas has to be done dependent on time and temperature. Generally there is a reciprocal relationship between the temperature of the heat treatment and the duration thereof. If the heat treatment is done at a relatively high temperature, a relatively short treatment period is sufficient, and vice versa. The heat treatment should be conducted for at least about 10 minutes, 30 minutes or 1 hour. For example, the temperature is at least 30°C, at least 40°C or at least 50°C. It should not fall below 20°C in order to avoid too long treatment periods. The treatments may be carried out at 40 to 70°C, particularly at 45 to 65°C for 5 minutes to 2 hours, at 40 to 60°C for 10 minutes to 5 hours, or at 30 to 50°C for 20 minutes to 20 hours, particularly 1 hour to 15 hours.

An industrially practical heat treatment may involve, for example, a period of 20 minutes at 50°C, 10 hours at 50°C or 12 hours at 38°C.

Naturally, it should be considered during the heat treatment that, dependent on the propellant gas used, there is an upper limit for the heating, which should not be exceeded for safety reasons. When using a mixture of isobutane/butane/propane, the heat treatment should be carried out for long periods (in an hourly range) at no more than about 50°C.

The heat treatment is preferably done by incubation, for example in a heating cabinet, a heating chamber or an incubation room. The heat treatment is particularly carried out after addition of the propellant gas. According to the invention, it is possible to subject the whole batch to a heat treatment before packaging and mass production. With larger batches, the heat treatment of the tins containing the foam cream may be carried out on pallets in heating chambers. Temperatures of 35 — 40°C for a period of 10 to 40 hours can be used. Alternatively, the foam cream can be first dosed or bottled and then subjected to the heat treatment.

A further possibility for producing a stable foam cream is the heating of the propellant gas before or during its addition to the hydrophilic and lipophilic components. This is in contrast to well known methods, wherein the propellant gas is feeded with cooling. The heating of the propellant gas is preferably done at 25 to 60°C, particularly 25 to 50°C or 25 to 40°C. It may be advantageous to heat the propellant gas to at least 30°C or 40°C.

Also, a pressure test of the bottled tins comprising the propellant gas may be used as a (partial) heat treatment according to the present invention. In such instances, the tins are conducted through a water bath heated to 40 to 50°C. The relevant residence times in the water bath can be deducted from the incubation time.

Preferably, the inventive stable foam cream is a two-phase system. In preferred embodiments, the lipophilic components comprise fatty acids and dimethyl polysiloxanes, and the hydrophilic components are selected from the group consisting of triethanol-amine, mono propylene glycol, glycerine, sorbitol, poly(ethylene glycol) and poly(vinyl pyrrolidone).

In a preferred embodiment, the inventive foam cream comprises C₁₀- to C₂₂-fatty acids, emulsifiers and co-emulsifiers.

In a particularly preferred embodiment, the inventive foam cream comprises

- 4 to 15 percent by weight of oil-in-water emulsifier,

- 1 to 10 percent by weight of fatty acid,

- 0.4 to 2.3 percent by weight moisturiser,

- 0.05 to 1 percent by weight skin care agent and

- water balancing to 100 percent by weight.

The inventive foam cream may additionally comprise

- 1 to 3 percent by weight of glyceryl stearate,

- 3 to 6 percent by weight cetearyl alcohol,

- 4 to 6 percent by weight of stearic acid,

- 0.5 to 2 percent of weigth of paraffin,

- 0.4 to 2.3 percent by weight of triceteareth-4-phosphate,

- 1.5 to 4 percent by weight propylene glycol,

- 1.3 to 4.2 percent by weight of glycerine,

- 1 to 3 percent by weight of cetyl-sarcosinate,

- 0.05 to 1 percent by weight of allantoin as

- water balancing to 100 percent by weight.

The stable foam cream may, as additional components, further comprise hydrating (moisture binding) substances like urea, ethoxy diglycol, sodium chloride, magnesium chloride, sorbit, dexpanthenol, sodium lactate.

Further preferred additives of the stable foam cream are clotrimazol, oak bark extract, sage, rosemary, arnica, aloe vera, panthenol and camphor. These additives significantly destabilise foam creams.

The inventive stable foam cream provides improved properties after the heat treatment, when compared to foam creams known in the prior art. Thus, the described phenomenon of collapsing of the foams substantially does not occur. Furthermore, the foam creams are stable over a long period of time, and are substantially resistant against exterior disturbing actions like shaking during transport or temperature fluctuations. The inventive foam cream is stable even if it contains substances which destabilize foam creams not treated in accordance with the present invention.

It is assumed that the foam creams treated in accordance with the invention undergo a structural change, which is responsible for the improved properties.

The inventive stable foam cream is, in a preferred embodiment, a cosmetic or medicinal foam cream, particularly a skin foam cream. It may contain commonly known components and additives for such skin creams, like those disclosed, for example, in WO-A-98/31339 or WO-A-99/08649.

The present invention also relates to a method of manufacture of a stable foam cream, comprising lipophilic and hydrophilic components and a propellant gas, which is characterized in that in this method a cream preparation comprising lipophilic and hydrophilic components is prepared, then propellant gas is added, so that a foam cream is formed, and the foam cream is subjected to a heat treatment and/or the propellant gas is heated before and/or during its addition to the cream preparation. In a further embodiment, the propellant gas is heated before and/or during its addition to the cream preparation.

In a preferred embodiment of the inventive method for the manufacture of the stable foam cream, the foam cream has collapsed or partially collapsed after addition of the propellant gas and before carrying out the heat treatment. This means that the inventive method does not only allow for a stabilization of foam creams, but also allows to reconstitute foam creams which have collapsed after addition of the propellant gas and typically can no longer be used by the manufacturer, by carrying out a heat treatment.

### EXAMPLES

Example 1

Manufacture of a Foam Cream

The foam cream is prepared in a closed apparatus which can be heated and cooled and which is equipped with a self-discharging homogenizer and a heatable dosing funnel. The manufacture of phase I is done in a heatable dosing funnel by melting a mixture comprising 2 weight percent of glyceryl stearate, 4 weight percent of cetearyl alcohol, 5 weight percent of stearyl acid, 1 weight percent of paraffin, 0.5 weight percent of triceteareth-4-phosphate, 5 weight percent of decyl oleate, 5 weight percent of octyl dodecanol and 0.2 weight percent of dimethicone at 75°C. This phase is metered under stirring to a phase II already present in a closed apparatus which can be heated and cooled and which comprises a self-discharging homogenizer. This phase II consists of an aqueous mixture comprising 5.5 weight percent of urea, 2.5 weight percent propylene glycol, 2.5 weight percent of glycerine, 2 weight percent of sodium lauroyl sarcosinate and 63 weight percent of water. Formation of a homogenous blend of both phases I and II has to be assured.

The metered addition of phase I is done at a temperature of 75°C. Both phases are combined while permanently stirring at a medium stirring speed, and a uniform homogenizing has to be assured. The temperature is held between 20 and 30 minutes at 75°C. Thereafter, 0.1 weight percent allantoin and 1.1 weight percent of triethanolamine (for adjusting the pH-value to a value between 7.8 and 8.0) are added. The resulting mixture is cooled to a temperature of from 30 to 40°C while stirring permanently.

After a temperature of 40°C has been reached, 0.5 weight percent of aloe vera and 0.1 weight percent of panthenol are added. The addition of these components significantly destabilizes foam creams and typically leads to a collapse of the foam cream.

It is stirred for a period sufficiently long enough to stabilize the pH-value and then the foam cream is filled into suitable storage containers or into the pressurized dispenser, respectively, with 9% isobutane/butane/propane.

Example 2

After filling of the foam cream of Example 1, heat treatment is carried out in that the filled containers are stored in a storage room at 38°C for 12 hours.

Example 3

The manufacture of a foam cream is carried out according to the method of Example 1. The propane/butane-mixture may be warmed to 25°C before adding.

Example 4

The preparation of a foam cream is carried out in accordance with the method as described in Example 1. After filling of the foam cream, the filled containers are heated for 20 minutes in a heating cabinet with an interior temperature of 50°C.

Example 5

The preparation of a foam cream is carried out in accordance with the method as described in Example 1, wherein the propane/butane-mixture to be added may be warmed to 25°C and the filled containers are stored after filling of the foam cream for 12 hours in a storage room at 38°C.

Example 6

A foam cream is prepared in accordance with Example 1, with the difference that instead of aloe vera and panthenol, clotrimazol is added to the mixture. The rest of the procedure is as described in Example 1. The addition of this component destabilizes foam creams to a large extent and typically results in a collapse of the foam cream. One part of the foam cream is subjected to a heat treatment in accordance with Example 2, another part is subjected to a heat treatment in accordance with Example 4.

Example 7

The same procedure as described in Example 6 is carried out, with the difference that instead of adding clotrimazol to the foam cream, oak bark extract is added. This additive can destabilize foam creams and typically leads to a collapse of the foam cream.

Example 8

The procedure as described in Example 6 is carried out, with the difference that instead of clotrimazol, sage is added to the foam cream. The addition of this component destabilizes foam creams to a large extent and typically leads to a collapse of the foam cream.

Example 9

The procedure as described in Example 6 is carried out, with the difference that instead of clotrimazol, rosemary is added to the foam cream. The addition of this component destabilizes foam creams to a large extent and typically leads to a collapse of the foam cream.

Example 10

The procedure as described in Example 6 is repeated, with the difference at instead of clotrimazol, arnica is added to the foam cream. The addition of this component destabilizes foam creams to a large extent and typically leads to a collapse of the foam cream.

Example 11

The procedure as described in Example 6 is repeated, with the difference that camphor is added to the foam cream instead of clotrimazol. The addition of this component destabilizes foam creams to a large extent and typically leads to a collapse of the foam cream.

Results:

The foam creams in accordance with Examples 2 to 11, which were subjected to a heat treatment are stable. A collapse of these creams could not be observed. The foam creams in accordance with Example 1 without a heat treatment, however, tend to collapse.

What is Claimed is:

1. A method for the manufacture of a stable foam cream comprising lipophilic and hydrophilic components and a propellant gas, characterized in that in said method a cream preparation comprising lipophilic and hydrophilic components is prepared,
- then, propellant gas is added so that a foam cream is formed and the foam cream is subjected to a heat treatment,
- and/or the propellant gas is heated before and/or during the addition to the cream preparation,
- wherein the heat treatment is carried out for at least one hour at at least 30°C, or for at least 5 minutes at at least 40°C.

2. Method according to claim 1, characterized in that the foam cream is provided as a two phase system.

3. Method according to any one of claims 1 or 2, characterized in that the lipophilic components are selected from fatty acids and dimethyl polysiloxanes, and the hydrophilic components are selected from the group consisting of triethanol amine, mono propylene glycol, glycerine, sorbitol, poly(ethylene glycol) and poly(vinyl pyrrolidone).

4. Method according to any one of claims 1 to 3, characterized in that the foam cream comprises C₁₀-to C₂₂-fatty acids, emulsifiers and coemulsifiers.

5. Method according to any one of claims 1 to 4, characterized in that the foam cream comprises
- 4 to 15 percent by weight of oil-in-water emulsifier,
- 1 to 10 percent by weight of fatty acid,
- 0.4 to 2.3 percent by weight of moisturiser,
- 0.05 to 1 percent by weight of skin care agent, and
- water balancing to 100 percent by weight.

6. Method according to any one of claims 1 to 5, characterized in that the foam creams comprises
- 1 to 3 percent by weight of glyceryl stearate,
- 3 to 6 percent by weight cetearyl alcohol,
- 4 to 6 percent by weight of stearic acid,
- 0.5 to 2 percent of weigth of paraffin,
- 0.4 to 2.3 percent by weight of triceteareth-4-phosphate,
- 1.5 to 4 percent by weight propylene glycol,
- 1.3 to 4.2 percent by weight of glycerine,
- 1 to 3 percent by weight of cetyl-sarcosinate,
- 0.05 to 1 percent by weight of allantoin and
- water balancing to 100 percent by weight.

7. Method according to any one of claims 1 to 6, characterized in that the foam cream additionally comprises hydrating (moisture binding) substances like urea, ethoxy diglycol, sodium chloride, magnesium chloride, sorbit, dexpanthenol, sodium lactate and/or additives like clotrimazol, oak bark extract, sage, rosemary, arnica, aloe vera, panthenol and/or camphor.

8. Method according to any one of claims 1 to 7, characterized in that the foam cream collapses, or partly collapses, after addition of the propellant gas and before conducting the heat treatment.

9. Stable foam cream, obtainable by a method according to any one of claims 1 to 8.

10. Stable foam cream according to claim 9, comprising lipophilic and hydrophilic components and a propellant gas, obtainable by subjecting the foam cream to a heat treatment, characterized in that the heat treatment is carried out for a time period of at least 5 minutes and/or for 5 minutes to 20 hours at 30 to 75°C.

11. Stable foam cream according to claims 9 or 10, characterized in that the heat treatment is conducted after addition of the propellant gas.

12. Stable foam cream according to one of claims 9 to 11, characterized in that the foam cream is a two phase system.

13. Stable foam cream according to any one of claims 9 to 12, characterized in that the lipophilic components are selected from fatty acids and dimethyl polysiloxanes and the hydrophilic components are selected from the group consisting of triethanol amine, mono propylene glycol, glycerine, sorbitol, poly(ethylene glycol) and poly(vinyl pyrrolidone).

14. Stable foam cream according to any one of claims 9 to 13, characterized in that the foam cream comprises C₁₀-to C₂₂-fatty acids, emulsifiers and coemulsifiers.

15. Stable foam cream according to any one of claims 9 to 14, characterized in that the foam cream comprises
- 4 to 15 percent by weight of oil-in-water emulsifier,
- 1 to 10 percent by weight of fatty acid,
- 0.4 to 2.3 percent by weight moisturiser,
- 0.05 to 1 percent by weight skin care agent and
- water balancing to 100 percent by weight.

16. Stable foam cream according to any one of claims 9 to 15, characterized in that the foam cream comprises
- 1 to 3 percent by weight of glyceryl stearate,
- 3 to 6 percent by weight cetearyl alcohol,
- 4 to 6 percent by weight of stearic acid,
- 0.5 to 2 percent of weigth of paraffin,
- 0.4 to 2.3 percent by weight of triceteareth-4-phosphate,
- 1.5 to 4 percent by weight propylene glycol,
- 1.3 to 4.2 percent by weight of glycerine,
- 1 to 3 percent by weight of cetyl-sarcosinate,
- 0.05 to 1 percent by weight of allantoin, and
- water balancing to 100 percent by weight.

17. Stable foam cream according to any one of claims 9 to 16, characterized in that the foam cream additionally comprises hydrating (moisture binding) substances like urea, ethoxy diglycol, sodium chloride, magnesium chloride, sorbit, dexpanthenol, sodium lactate and/or additives like clotrimazol, oak bark extract, sage, rosemary, arnica, aloe vera, panthenol and/or camphor.
